(19) United States
(12) Patent Application Publication
(10) Pub. No.: US 2019/0156942 A1
(43) Pub. Date: May 23, 2019

(54) APPARATUS FOR ASSESSING MEDICAL DEVICE QUALITY

(71) Applicant: KONINKLIJKE PHILIPS N.V., EINDHOVEN (NL)

(72) Inventors: Serverius Petrus Paulus PRONK, VUGHT (NL); Carolina RIBBING, AACHEN (DE); Johannes Henricus Maria KORST, EINDHOVEN (NL); Mauro BARBIERI, EINDHOVEN (NL); Marc Andre PETERS, VELDHOVEN (NL); Qi GAO, EINDHOVEN (NL); Reza KARIMI, EINDHOVEN (NL)

(21) Appl. No.: 16/316,746

(22) PCT Filed: Jul. 17, 2017
(86) PCT No.: PCT/EP2017/067975

§ 371 Date: Jan. 10, 2019

(30) Foreign Application Priority Data
Jul. 15, 2016 (EP) 16179592.7

(51) Int. Cl. G16H 40/40; G16H 40/63

(52) U.S. Cl. CPC G16H 40/40; G16H 40/63

(57) ABSTRACT

The present invention relates to an apparatus for assessing medical device quality. It is described to provide (210) at least one report associated with at least one medical image acquired by a medical device, wherein a report is associated with a corresponding medical image. The at least one report is analyzed and at least one image quality parameter is generated (220), wherein an image quality parameter is associated with a 5 corresponding report. The at least one image quality parameter is assessed and generating alert information relating to the medical device is generated (230).

### FIELD OF THE INVENTION

The present invention relates to an apparatus for assessing medical device quality, to a system for providing medical device alerts, to a method for assessing medical device quality, as well as to a computer program element and a computer readable medium.

### BACKGROUND OF THE INVENTION

The general background of this invention is the field of determining the quality of a medical device. Cost-effective use of medical imaging modalities requires high system availability. Typically hospitals have contracts with maintenance service providers. Most maintenance is planned or corrective maintenance. For the latter case, when a system malfunctioning is detected, a call is made to a service center where a service engineer starts troubleshooting.

US 2013/0251219 A1 describes that a medical image quality reporting and monitoring system for use in a medical imaging system comprises a medical image computer including a display processor, a display and a report generator. The display processor generates data representing an image for display including a user selectable image element enabling a user to identify at least one medical image as having an image quality deficiency. The display presents the image. The report generator, in response to detection of selection of the image element, identifying at least one medical reduced quality image as having an image quality deficiency, automatically generates a report. The report comprises, data representing an anonymized reduced quality image having the image quality deficiency, a time of acquisition of the reduced quality image and imaging system acquisition settings used in acquiring the reduced quality image.

### SUMMARY OF THE INVENTION

It would be advantageous to have improved apparatus for assessing medical device quality, and an improved system for providing medical device alerts using such an apparatus. The object of the present invention is solved with the subject matter of the independent claims, wherein further embodiments are incorporated in the dependent claims. It should be noted that the following described aspects and examples of the invention apply also for the apparatus for assessing medical device quality, the system for providing medical device alerts, the method for assessing medical device quality, and for the computer program element and the computer readable medium.

According to a first aspect, there is provided an apparatus for assessing medical device quality, comprising:

an input unit; and

a processing unit.

The input unit is configured to provide the processing unit with at least one report associated with at least one medical image acquired by a medical device, wherein a report is associated with a corresponding medical image. The processing unit is configured to implement a classifier module to analyze the at least one report and generate at least one image quality parameter, wherein an image quality parameter is associated with a corresponding report. The classifier module is configured to apply a natural language algorithm to the at least one report to generate the at least one image quality parameter. The natural language algorithm comprises a learning algorithm, and the learning algorithm is configured to generate a value for an image quality parameter on the basis of at least one training data. The processing unit is also configured to implement an assessment module to assess the at least one image quality parameter and generate alert information relating to the medical device.

In this manner, an image quality parameter is derived from reports associated with images acquired by a medical device, and this is used to generate alert information. This enables predictive maintenance of the medical device to be made, and can also be used as background information for the device when routine scheduled maintenance is made, as well as aiding troubleshooting if a call is made to a service center for example.

In other words, natural language as words being used for example by a radiologist when preparing reports on medical imagery can be analyzed, thereby providing an effective way of using reports to determine information relating to the medical device.

In other words, the learning algorithm is trained using reports (such as radiology reports) in which text fragments pertaining to image quality issues can have been manually labelled as such (supervised learning).

In this way, the accuracy of the generation of the image quality parameter can be continually improved as more reports are analyzed. For example, through the periodic manual labelling of reports as described above, verifying that there was such an image problem identified by a radiologist—this labelling can be carried out by an image technician or a technician familiar with the medical device, and thereby the learning algorithm is improved through operating on such “supervised” input data.

Data protection issues relating to analysis of patient imagery can be problematic, even when processing to anonymize that imagery is made, and the display and processing of imagery requires significant computational resource and can be difficult. However, by deriving an image quality parameter from reports associated with images acquired by a medical device, such data protection of the analysis of imagery is not at issue and processing is made more efficient and simpler.

Thus, in this way the apparatus automatically detects when an image deficiency has been detected. As this is less obtrusive for a radiologist, he can continue the assessment of an image.

In this way, information relating to the image quality provided by a medical device is extracted to aid in the assessment of the functioning of the medical device.

This enables the functioning of the medical device to be determined, on the basis of reports, without the requirement to undertake extensive image quality analysis of images to identify artefacts in those images indicative of problems with the medical device.

Additionally, assessment of the functioning of the medical device being based on reports, means that the extensive analysis of imagery is not required to identify device problems, where such remote analysis of imagery can be problematic due to data protection issues.

The apparatus has the benefit that medical device quality can be assessed, without any image processing having to be carried out. Also, image quality parameters from the medical device are not required in order to assess the quality of the medical device.

In an example, a report of the at least one report comprises text and wherein the classifier module is configured to analyze at least one portion of the text to generate an image quality parameter.

In other words, a report or reports associated with a medical image(s) is an analyzed using natural language processing pertaining to the quality of individual images, enabling image quality parameters to be generated that for example can signal the presence of artefacts in the image(s), with alert information being generated that is useable to generate early alerts relating to the medical device.

In this manner, a computationally efficient processing of the report to generate image quality parameters can be implemented.

In an example, the classifier module is configured to generate the image quality parameter on the basis of an image quality issue in the at least one portion of text.

In other words, text relating to imaging issues can be analyzed and used to provide information relating to the performance of the medical device, thereby providing an effective and computationally efficient way of monitoring and assessing the integrity of the medical device.

In this manner, an effective and simple manner of analyzing a text report is provided in order to determine the magnitude of image related issues.

In an example, the classifier module is configured to use a database containing a plurality of text fragments, and the classifier module is configured to compare the at least one portion of text to at least one of the plurality of text fragments to generate the image quality parameter.

This enables the image quality parameter to be generated in a simple and efficient manner, and enables a learning process to be implemented, for example where the database is augmented with text fragments over time.

In an example, the at least one report comprises a plurality of reports and wherein the at least one quality parameter comprises a plurality of quality parameters, and wherein the assessment module is configured to generate alert information on the basis of a determined trend in the plurality of quality parameters.

In this manner, when the trend in the quality parameters indicates a growing (e.g. severe) issue, this can be captured enabling remedial action to be taken before the issue effects the imaging quality of the medical device or the issue indicates a problem with the medical device, which if left unattended could be more expensive to resolve than addressing it now before it becomes a problem.

In an example, the at least one report is generated by at least one user on the basis of the at least one medical image.

In this manner, taking the example of the medical device being an X-ray device, by analyzing reports made by a radiologist relating to medical imagery, the functioning of the X-ray imaging device can be assessed. This equally applies to MRI devices, to CT devices, to ultrasound devices etc.

In an example, the assessment module is configured to generate the alert information based on a comparison of the at least one image quality parameter with a threshold.

In this manner, an efficient means is provided to determine if there is a problem (or developing problem) with the medical device that requires rectification.

In an example, the at least one report comprises a plurality of reports and wherein the at least one quality parameter comprises a plurality of quality parameters, and wherein the assessment module is configured to generate the alert information based on a number of the plurality of quality parameters exceeding the threshold.

In this way, a statistical analysis can be provided in order that noise in imagery does not lead to the generation of alert information that indicates a problem. Also, alert information is generated that enables a customer service ticket to be issued if the reports indicate that there is a persistent issue in the imagery acquired by the medical device, such as persistent low quality.

According to a second aspect, there is provided a system for providing medical device alerts, comprising:

an information providing unit;

an apparatus for assessing medical device quality according to the first aspect; and

an output unit.

The at least one report is provided from the information providing unit to the input unit. The processing unit is configured to generate alert information on the basis of the at least one report provided from the information providing unit. The output unit is configured to output an alert based on the alert information.

In this manner service alerts can be output automatically, for example to a remote service center, which can if necessary schedule maintenance of the medical device, through for example a service visit.

According to a third aspect, there is provided a method for assessing medical device quality, comprising:
b) providing at least one report associated with at least one medical image acquired by a medical device, wherein a report is associated with a corresponding medical image;
c) analyzing the at least one report and generating at least one image quality parameter, wherein an image quality parameter is associated with a corresponding report; and
d) assessing the at least one image quality parameter and generating alert information relating to the medical device.

In an example the method comprises:
a) generating the at least one report by at least one user on the basis of the at least one medical image.

According to another aspect, there is provided a computer program element controlling apparatus as previously described which, if the computer program element is executed by a processing unit, is adapted to perform the method steps as previously described.

According to another aspect, there is provided a computer readable medium having stored computer element as previously described.

Advantageously, the benefits provided by any of the above aspects equally apply to all of the other aspects and vice versa.

The above aspects and examples will become apparent from and be elucidated with reference to the embodiments described hereinafter.

### BRIEF DESCRIPTION OF THE DRAWINGS

Exemplary embodiments will be described in the following with reference to the following drawings:

FIG. 1 shows a schematic set up of an example of an apparatus for assessing medical device quality;

FIG. 2 shows a schematic set up of an example of a system for providing medical device alerts;

FIG. 3 shows a method for assessing medical device quality;

FIG. 4 shows a detailed architecture of a system for providing medical alerts along with an adjoining picture archiving and communication system (PACS) and an adjoining common analyzer tool and early alert system.

### DETAILED DESCRIPTION OF EMBODIMENTS

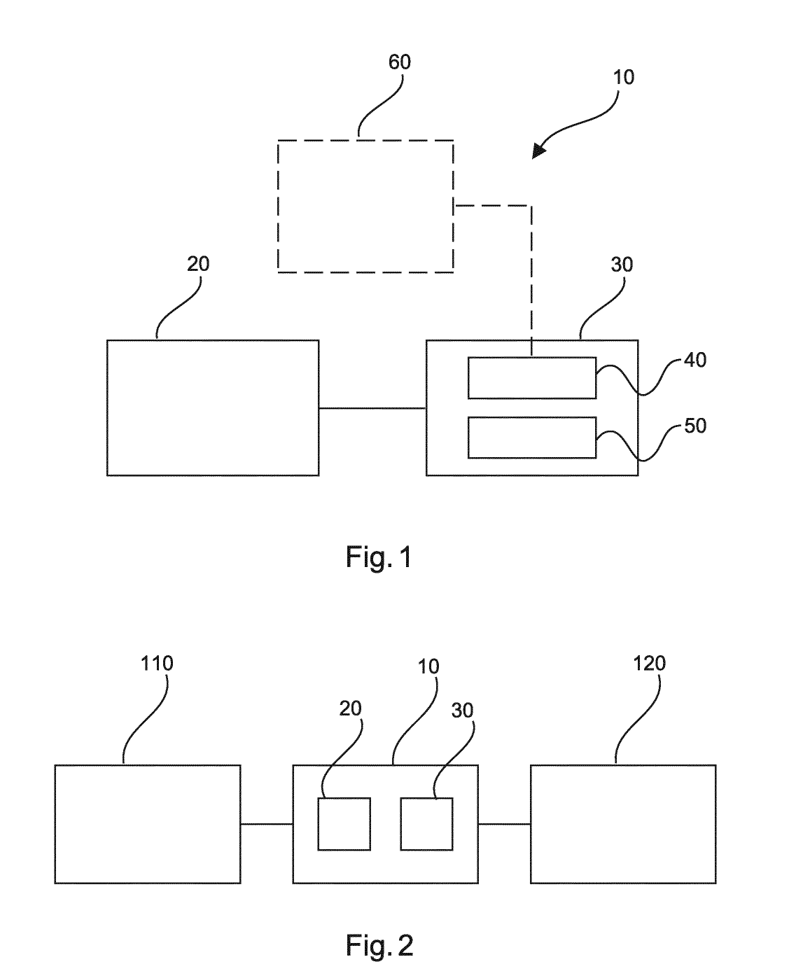

FIG. 1 shows an example of an apparatus 10 for assessing medical device quality. The apparatus 10 comprises an input unit 20, and a processing unit 30. The input unit 20 is configured to provide the processing unit 30 with at least one report associated with at least one medical image acquired by a medical device, wherein a report is associated with a corresponding medical image. The processing unit 30 is configured to implement a classifier module 40 to analyze the at least one report and generate at least one image quality parameter, wherein an image quality parameter is associated with a corresponding report. The processing unit 30 is also configured to implement an assessment module 50 to assess the at least one image quality parameter and generate alert information relating to the medical device.

In an example, each report is associated with a separate medical image. In other words, as medical images are acquired a change in the quality of the medical images can be determined and correlated with the medical device in order to effect remedial action.

In an example, reports can be associated with one medical image. In other words, the quality could vary across an image being of excellent quality at one side of the image and acceptable in the middle of the image and unacceptable at the other side of the image, and this information can be used to effect remedial action. Thus, here reports can mean a number of separate items of information relating to an image or can mean items of information within a single source relating to an image. In other words, reports can mean, as described above, in a single “report”, different information being provided relating to an image such as comments on the quality at different areas of the image.

In an example, the classifier module is configured to generate a numerical value for the image quality parameter.

In this manner, a simple to analyze measure of quality is provided in the form of a number, thereby enabling the image quality associated with one report to be easily and simply compared and contrasted with the image quality associated with another report.

In an example, the at least one image quality parameter is useable to signal the presence of at least one artefact in the at least one medical image.

In an example, the classifier module is configured to distinguish between positive and negative reports. By teaching the classifier to distinguish between positive reports, i.e., reports associated with images of good quality, and negative reports, i.e., reports associated with images of bad quality, based on a number of positive and negative example reports, it automatically adapts to the radiologist. That is to say, if a radiologist usually expresses himself in a negative sense, then this will probably be present in both the positive and negative examples. As long as the classifier can make a distinction, there will be no inherent problem in dealing with personal taste. In this sense, the classifier personalizes itself to the radiologist.

According to an example, a report of the at least one report comprises text and the classifier module 40 is configured to analyze at least one portion of the text to generate an image quality parameter.

In an example, the at least one portion of the text is the same as the text of the report.

In an example, each report of the at least one report comprises text, that can be different for different reports, and the classifier module is configured to analyze at least one portion of the text in the different reports to generate image quality parameters, that can be different for different reports, for the different reports.

In an example, analysis of the at least one portion of the text comprises parsing of the at least one portion of the text.

In an example, the classifier module is configured to generate the at least one portion of text from the text. In an example, the classifier module is configured to remove stop words from the text when generating the at least one portion of text. For example, the text string “the image contrast is too low” can be processed by the classifier module to extract features such as: “image”; “contrast”; “low”. In an example, processed features are further sub-divided to form n-grams such as: “ima”; “mag”; “age”; “con”; “ont”.

According to an example, the classifier module 40 is configured to generate the image quality parameter on the basis of an image quality issue in the at least one portion of text.

In an example, a positive count is associated with the at least one portion of text if it has been determined as pertaining to an image quality issue.

In an example, a negative count is associated with the at least one portion of text if it has been determined as not pertaining to an image quality issue.

In this way, the classifier module can make a distinction between positive and negative images, and hence assess medical device quality, on the basis of negative comments made by a radiologist in a report associated with an image.

According to an example, the classifier module 40 is configured to use a database 60 containing a plurality of text fragments. The classifier module 40 is configured to compare the at least one portion of text to at least one of the plurality of text fragments to generate the image quality parameter.

In an example, the database contains text fragments that are known to relate to image quality issues (e.g., “low contrast”; “blocks”, “dots”; “stripes”; “smear”; “hazy”; “fuzzy”; “poor”). In an example, the database comprises a dictionary. In other words, a dictionary of terms used to describe problems or issues can be provided, and a text fragment in the report can be compared to or with the terms in the dictionary to generate the image quality parameter in order to quantify the issue. In an example, the dictionary contains both positive and negative text fragments to aid in the learning process. Positive text fragments are, for example “high contrast”, “sharp image”, et cetera. If only negative text fragments are available, then the absence of such text fragments may indicate no image quality issue. However, by providing the ability to process both positive and negative text fragments, the learning process is improved.

According to an example, the classifier module 40 is configured to apply a natural language algorithm to the at least one report to generate the at least one image quality parameter.

According to an example, the natural language algorithm comprises a learning algorithm. The learning algorithm is configured to generate a value for an image quality parameter on the basis of at least one training data.

In an example, the training data is derived from medical images that have associated reports, and where issues associated with the images have been quantified or verified. In this manner, the correlation information forms a dataset against which the learning algorithm can operate.

In an example, the at least one training data is derived from at least one correlation information relating to at least one determined quality.

In an example, the correlation information is derived from medical images that have associated reports, and where issues associated with the images have been quantified or verified. In this manner, the correlation information forms a dataset against which the learning algorithm can operate.

In an example, the learning algorithm is configured to generate a value for an image quality parameter on the basis of the whole text associated with a report. In an example, the learning algorithm is configured to determine the identity of the radiologist from the text associated in the report. This means that the learning algorithm can differentiate one radiologist from another on the basis of the language used by different radiologist to identify the radiologist, and this is what one meaning of “identify” means. However, example language for different radiologists in a database can then be referenced to enable this differentiation to provide information relating to the probability that the radiologist was a particular radiologist, and this is what another meaning of “identify” means. In this way, the learning algorithm is able to determine an overall probability score for a report relating to an image quality parameter relating to the overall negative or positive undertone of the language used in the report, and can take into account the identity of the radiologist in this process. For example, different radiologists may described the same image differently in terms of different uses of negative language even though they equally appreciate what the issue is in the image and its severity. In an example, the learning algorithm can be aided in this process, through a trained expert radiologist and/or medical device expert reviewing the imagery and providing baseline report information relating to the imagery. This then enables the comments from different radiologists to be quantified in terms of how the language used by the radiologist relates to issues.

In an example, if the report contains no negative comments, the learning algorithm is configured to determine a probability of negativity (or positivity) from the whole report, and this is used to further develop the learning algorithm and to provide information relating to the medical device.

In an example, an entire, labelled, report can be used as a text fragment, whereby pieces of text (words, n-grams) that do not directly or obviously relate to image quality can also be used in the learning process. This make personalization to the radiologist possible, thereby permitting the apparatus to automatically take into account the identity of the user providing the report, and in this manner the accuracy of the assessment of medical device quality can be improved as differences in how different users quantify the same or similar imaging problems can be mitigated.

In this manner, by using a learning algorithm, that can be used together with a dictionary, enables the apparatus to learn from the annotations by the radiologist, aiming to further improve the automatic detection.

In an example, the learning algorithm is provided with a set of reports associated with associated medical images, and the learning algorithm scans the set of reports to learn (develop) a (statistical) model that is used to generate the at least one image quality parameter for the at least one report that has not previously been presented to the apparatus. In an example, the output of a machine learning algorithm is a probability that indicates how probable it is that the image has a quality issue. For example, in a naive Bayesian classifier, which computes, based on the training examples and using prior and conditional probabilities, a positive posterior probability. This positive posterior probability fulfills the role mentioned above. Similar probabilistic output is generally possible with other classifiers.

As an enhancement to the learning process, in an example, when the positive posterior probability is close to the negative posterior probability, i.e., they are both close to 0.5, the apparatus asks for an explicit image quality assessment of the radiologist and adds this to the training set of positives and negatives.

According to an example, the at least one report comprises a plurality of reports and wherein the at least one quality parameter comprises a plurality of quality parameters. The assessment module 50 is configured to generate alert information on the basis of a determined trend in the plurality of quality parameters.

According to an example, the at least one report is generated by at least one user on the basis of the at least one medical image.

In an example, a report of the at least one report is generated through the processing of spoken input from a radiologist. In this way, a radiologist is able to verbally describe images, and reports are generated that can be processed by the apparatus in order to assess medical device quality.

According to an example, the assessment module 50 is configured to generate the alert information based on a comparison of the at least one image quality parameter with a threshold.

According to an example, the at least one report comprises a plurality of reports and wherein the at least one quality parameter comprises a plurality of quality parameters. The assessment module 50 is configured to generate the alert information based on a number of the plurality of quality parameters exceeding the threshold.

In an example, the at least one report is associated with medical data acquired by any one or more than one of the following medical devices: X-ray device; MRI device; PET device; CT device; or ultrasound device.

FIG. 2 shows an example of a system 100 for providing medical device alerts. The system 100 comprises an information providing unit 110, an apparatus 10 for assessing medical device quality according to any of the examples described in relation to FIG. 1, and an output unit 120. The at least one report is provided from the information providing unit 110 to the input unit 20. The processing unit 30 is configured to generate alert information on the basis of the at least one report provided from the information providing unit 110. The output unit 120 is configured to output an alert based on the alert information.

In an example, the information providing unit is comprised within the medical device. In other words, the medical device can acquire imagery and a part of that medical device enables a radiologist to generate at least one report relating to imagery, or the report is created and stored in the medical device.

In an example, the information providing unit is an information storage device.

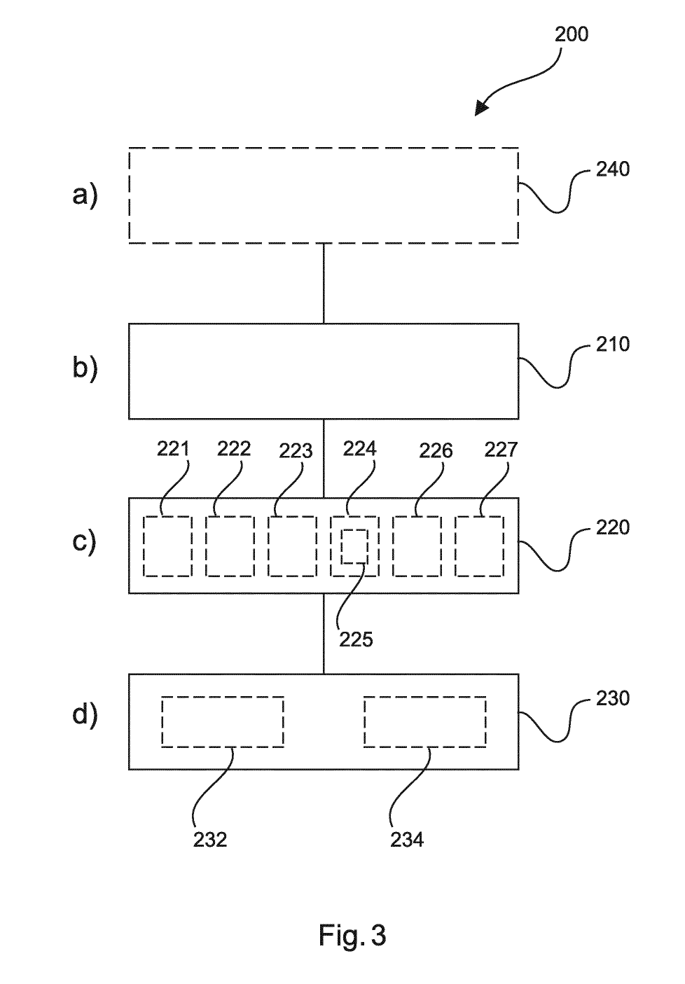

FIG. 3 shows a method 200 for assessing medical device quality in its basic steps. The method 200 comprises:

in a providing step 210, also referred to as step b), at least one report is provided associated with at least one medical image acquired by a medical device, wherein a report is associated with a corresponding medical image;

in an analyzing and generating step 220, also referred to as step c), the at least one report is analyzed and at least one image quality parameter is generated, wherein an image quality parameter is associated with a corresponding report; and

in an assessing and generating step 230, also referred to as step d), the at least one image quality parameter is assessed and alert information relating to the medical device is generated.

In an example, step c) comprises generating 221 a numerical value for the image quality parameter.

In an example, a report of the at least one report comprises text and wherein step c) comprises analyzing 222 at least one portion of the text to generate an image quality parameter.

In an example, step c) comprises generating 223 the image quality parameter on the basis of an image quality issue in the at least one portion of text.

In an example, step c) comprises using 224 a database containing a plurality of text fragments, and step c) further comprises comparing 225 the at least one portion of text to at least one of the plurality of text fragments to generate the image quality parameter.

In an example, the database comprises a dictionary. In an example, the dictionary comprises two parts, one part relating to negative words and a second part relating to positive words. This enables the dictionary to be built and maintained more easily.

In an example, step c) comprises applying 226 a natural language processing algorithm to the at least one report to generate the at least one image quality parameter.

In an example, the natural language algorithm comprises a learning algorithm, and wherein step c) comprises using 227 the learning algorithm to generate a value for an image quality parameter on the basis of at least one training data.

In an example, the at least one report comprises a plurality of reports and wherein the at least one quality parameter comprises a plurality of quality parameters, and wherein step d) comprises generating alert information on the basis of a determined trend in the plurality of quality parameters.

According to an example, the method comprises: in a generating step 240, also referred to as step a), the at least one report is generated by at least one user on the basis of the at least one medical image.

In an example, step d) comprises generating 232 the alert information based on a comparison of the at least one image quality parameter with a threshold.

In an example, the at least one report comprises a plurality of reports and wherein the at least one quality parameter comprises a plurality of quality parameters, and wherein step d) comprises generating 234 the alert information based on a number of the plurality of quality parameters exceeding the threshold.

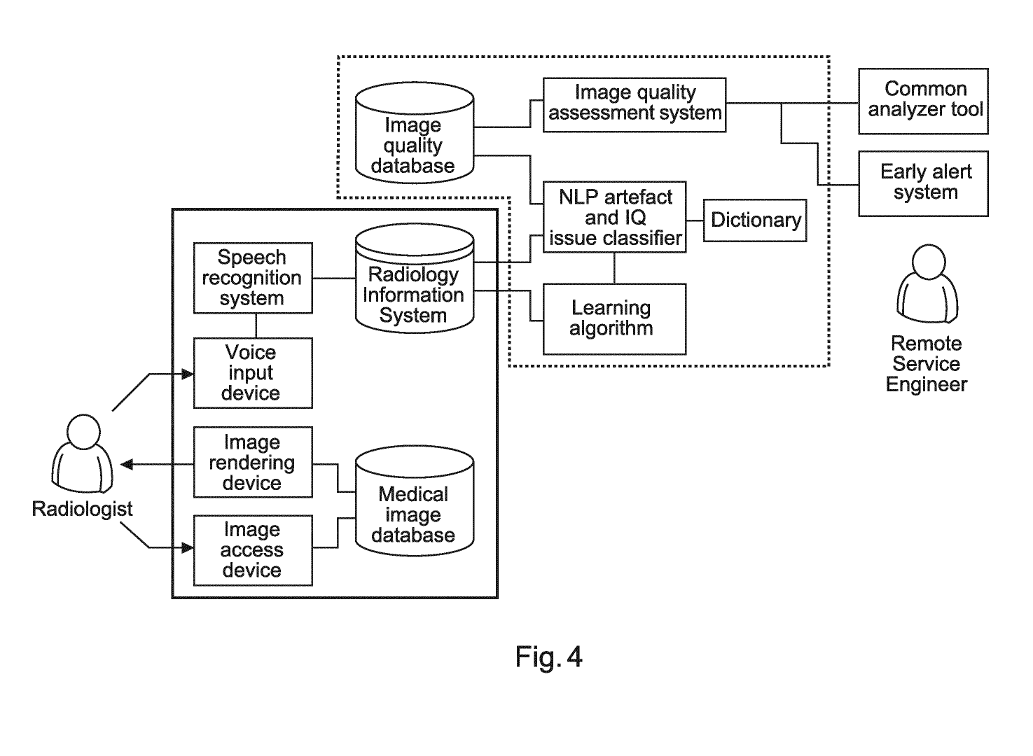

The apparatus and method for assessing medical device quality and the system for providing medical device alerts are now described in more detail in conjunction with FIG. 4.

FIG. 4 shows a detailed architecture of the environment within which an example of the system for providing medical device alerts operates, and where the system makes use of an example of the apparatus, and an example of the method, for assessing medical device quality. The features bounded by the solid line are a picture archiving and communication system (PACS), with an example of the system for providing medical device alerts bounded by the dashed lines. The system for providing medical device alerts is in communication connection with a common analyzer tool and with an early alert system that a remote service engineer has access to. The system for providing medical device alerts is also in communication connection with a radiology information system of the PACS.

Referring to FIG. 4, a radiologist has access to an image database to view the images contained therein and to write one or more radiology reports, possibly using voice input. Each image may have one, or a number of reports associated with it, and some images may not have a report associated with it. The report contains information relating to the quality of the image, such as describing that parts of the image are out of focus, have low contrast, are poor, or conversely are very precise and good, or adequate. In other words, the report contains information relating to the images that a radiologist uses to describe images and the quality of those images. This input is analyzed using speech recognition and stored as text in a database of a Radiology Information System (RIS). A natural language processing (NLP) system analyses the textual radiology reports to extract information related to the quality of an image. The radiologist can write the radiology report in text manually, rather than using speech, with the NLP analyzing the radiology reports in the same manner. The NLP system can make use of a dictionary that has been designed especially to aid in finding this information. An image quality assessment system analyses the image quality data to provide standardized input to, and that is compatible with, a common analyzer tool and an early alert system. The learning algorithm uses the annotations to learn and in this way improve the performance of the NLP system.

With continual reference to FIG. 4, specific details are:

The radiologist retrieves medical images from the Medical image database and creates a textual radiology report that is stored in the database of the Radiology Information System.

An NLP artifact and IQ issue classifier reads the textual radiology report and classifies text fragments using a Learning algorithm previously trained on reports annotated for image quality issues. The NLP artifact and IQ issue classifier can make use of a Dictionary containing text fragments that are known to relate to image quality issues (e.g. ‘low contrast’, ‘blocks’, ‘dots’, ‘stripes’, etc.).

The output of the NLP artifact and IQ issue classifier is a set of numerical scores pertaining to the quality of the image. These scores are stored in an Image quality database from which they are assessed by the Image quality assessment system to generate early alerts in case there are severe or growing issues with the image quality. A remote service engineer is notified by the Early alert system when the Image quality assessment system generates an alert. Remote service engineers can also access the Image quality assessment system on demand when performing troubleshooting e.g. using the Common analyzer tool.

### Learning Algorithm

The learning algorithm is trained using radiology reports in which text fragments pertaining to image quality issues have been manually labelled as such (supervised learning). Thus, text fragments stored in a dictionary are labeled with varying degrees of positivity and negativity. The learning algorithm is a machine learning algorithm such as a neural network, a random forest, a support vector machine. Other machine learning algorithms can be used.

For example, given a text fragment such as ‘the image contrast is too low’, the learning algorithm first removes stop words and extracts features such as: ‘image’; ‘contrast’; ‘low’ (typically the terms are further subdivided into n-grams such as: ‘ima’; ‘mag’; age’; ‘con’; ‘ont’). Then the learning algorithm associates to these features a positive count if the text fragment has been labelled as pertaining to image quality issues or a negative count if not. By scanning a large set of annotated fragments, the learning algorithm learns a (statistical) model that can then be applied to previously unseen text fragments to produce a numerical score pertaining to image quality issues.

### Other Uses

The information from the system for providing medical alerts (which can be considered to be an image quality assessment system) could also be used for other purposes than as alerts for service, e.g. for customer stratification for individualized service levels, comparison of sites/specialization fields/regions, at new system introductions in field. Other uses, of the apparatus and method for assessing medical device quality and the system for providing medical alerts, are: to detect whether the medical device is used properly to achieve the best image quality; to provide feedback to the technician or radiology department managers; and can even be used by the system manufacturer or service company to send experts to calibrate, or better set-up, the medical device to train the technicians using the medical device. From the above described embodiments, the skilled person would clearly appreciate how to implement these other uses.

In another exemplary embodiment, a computer program or computer program element is provided that is characterized by being configured to execute the method steps of the method according to one of the preceding embodiments, on an appropriate system.

The computer program element might therefore be stored on a computer unit, which might also be part of an embodiment. This computing unit may be configured to perform or induce performing of the steps of the method described above. Moreover, it may be configured to operate the components of the above described apparatus and/or system. The computing unit can be configured to operate automatically and/or to execute the orders of a user. A computer program may be loaded into a working memory of a data processor. The data processor may thus be equipped to carry out the method according to one of the preceding embodiments.

This exemplary embodiment of the invention covers both, a computer program that right from the beginning uses the invention and computer program that by means of an update turns an existing program into a program that uses invention.

Further on, the computer program element might be able to provide all necessary steps to fulfill the procedure of an exemplary embodiment of the method as described above.

According to a further exemplary embodiment of the present invention, a computer readable medium, such as a CD-ROM, USB stick or the like, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. An apparatus for assessing medical device quality, comprising:
an input unit; and
a processing unit;
wherein, the input unit is configured to provide the processing unit with at least one report associated with at least one medical image acquired by a medical device, wherein a report is associated with a corresponding medical image;
wherein, the processing unit is configured to implement a classifier module to analyze the at least one report and generate at least one image quality parameter, wherein an image quality parameter is associated with a corresponding report, wherein the classifier module is configured to apply a natural language algorithm to the at least one report to generate the at least one image quality parameter, wherein the natural language algorithm comprises a learning algorithm, wherein the learning algorithm is configured to generate a value for an image quality parameter on the basis of at least one training data; and
wherein, the processing unit is configured to implement an assessment module to assess the at least one image quality parameter and generate alert information relating to the medical device.

2. The apparatus according to claim 1, wherein a report of the at least one report comprises text and wherein the classifier module is configured to analyze at least one portion of the text to generate an image quality parameter.

3. The apparatus according to claim 2, wherein the classifier module is configured to generate the image quality parameter on the basis of an image quality issue in the at least one portion of the text.

4. The apparatus according to claim 2, wherein the classifier module is configured to use a database containing a plurality of text fragments, and the classifier module is configured to compare the at least one portion of the text to at least one of the plurality of text fragments to generate the image quality parameter.

5. The apparatus according to claim 1, wherein the at least one report comprises a plurality of reports and wherein the at least one quality parameter comprises a plurality of quality parameters, and wherein the assessment module is configured to generate alert information on the basis of a determined trend in the plurality of quality parameters.

6. The apparatus according to claim 1, wherein the at least one report is generated by at least one user on the basis of the at least one medical image.

7. The apparatus according to claim 1, wherein the assessment module is configured to generate the alert information based on a comparison of the at least one image quality parameter with a threshold.

8. The apparatus according to claim 7, wherein the at least one report comprises a plurality of reports and wherein the at least one quality parameter comprises a plurality of quality parameters, and wherein the assessment module is configured to generate the alert information based on a number of the plurality of quality parameters exceeding the threshold.

9. A system for providing medical device alerts, comprising:
an information providing unit;
an apparatus for assessing medical device quality according to preceding claim 1; and
an output unit;
wherein, the at least one report is provided from the information providing unit to the input unit;
wherein, the processing unit is configured to generate alert information on the basis of the at least one report provided from the information providing unit; and
wherein, the output unit is configured to output an alert based on the alert information.

10. A method for assessing medical device quality, comprising:
b) providing at least one report associated with at least one medical image acquired by a medical device, wherein a report is associated with a corresponding medical image;
c) analyzing the at least one report and generating at least one image quality parameter by applying a natural language processing algorithm comprising a learning algorithm to the at least one report and using the learning algorithm to generate a value for an image quality parameter on the basis of at least one training data, wherein the image quality parameter is associated with a corresponding report; and
d) assessing the at least one image quality parameter and generating alert information relating to the medical device.

11. The method according to claim 10, wherein the method comprises:
a) generating the at least one report by at least one user on the basis of the at least one medical image.

12. A computer program for controlling an apparatus which when executed by a processor is configured to carry out the method of claim 10.

13. A computer readable medium having stored the program element of claim 12.
